(19) United States
(12) Patent Application Publication
(10) Pub. No.: US 2010/0022894 A1
(43) Pub. Date: Jan. 28, 2010

(54) Intravascular Measurement

(75) Inventors: Michael Tittelbach, Nuernberg (DE); Claus Harder, Uttenreuth (DE)

Correspondence Address: BIOTECH BEACH LAW GROUP , PC, 5677 OBERLINE DRIVE, SUITE 204, SAN DIEGO, CA, 92121, US

(73) Assignee: BIOTRONIK VI PATENT AG, Baar (CH)

(21) Appl. No.: 12/508,986

(22) Filed: Jul. 24, 2009

(30) Foreign Application Priority Data
Jul. 28, 2008 (DE) 10 2008 040 790.9

(51) Int. Cl. A61B 5/02

(52) U.S. Cl. 600/481

(57) ABSTRACT

An implantable sensor for measuring fluidic parameters with a surface acoustic wave transponder for detecting vasomotor quantities and one retaining stent attached to each of two opposite ends of the surface acoustic wave transponder.

### CROSS-REFERENCE TO RELATED APPLICATIONS

This application claims benefit of priority to German patent application number 10 2008 040 790.9, filed Jul. 28, 2008; the contents of which are herein incorporated by reference in their entirety.

### FIELD OF THE INVENTION

The invention relates to an implantable sensor for detection of fluidic characteristics of blood flow.

### BACKGROUND OF THE INVENTION

Implantable pressure sensors which can be placed in a blood vessel and allow a determination of the blood pressure are known in the prior art. The measurement results of such pressure sensors are used to monitor cardiac output of patients with cardiac insufficiency or similar conditions. Such a pressure sensor is disclosed in US 2002/0045921, for example.

Telemetry units are frequently used to read out the values determined by the implantable pressure sensor; for example, after activation of a magnetic switch situated in or connected to the pressure sensor by a reading head, these telemetry units transmit the measured data wirelessly. Such a telemetry unit presupposes a power supply by battery or induction, so such pressure sensors are preferably used together with implantable cardiac pacemakers, which increasingly have telemetry functions, allowing a treating physician to read out a variety of medical and operational technical data.

However, such sensors have the disadvantage that they hinder blood flow and also constitute a significant risk of thrombosis in addition to the exacerbating effect on circulation because cells or blood platelets or other solid components of blood may be deposited on such sensors. If such a deposit is entrained by the blood flow away from the pressure sensor, it may result in a hazardous health impairment. This risk is therefore counteracted by anticoagulants, i.e., administration of medication to prevent coagulation, although that also entails corresponding risks and adverse effects.

Furthermore, pressure sensors situated directly in the blood flow have the disadvantage that the deposits described above may lead to a considerable technical impairment of the sensor, even resulting in total failure.

There is therefore a demand for an implantable sensor that will overcome one or more of the aforementioned shortcomings of the prior art.

### SUMMARY OF THE INVENTION

The present invention provides an implantable sensor for measurement of fluidic parameters, having a surface acoustic wave transponder (SAW transponder) for detection of vasomotor parameters and having a retaining stent, which is attached at two opposite ends of the surface acoustic wave transponder. The term “retaining stent” here means a stent-like retaining element, although it need not have the radial strength desired with a traditional stent but instead serves only to secure the surface acoustic wave transponder on the wall of the blood vessel. In particular, the retaining stents may also be shorter than traditional stents and may have a much lower radial strength.

The invention provides that it is possible to determine blood pressure and blood flow via the vasomotor system of the blood vessel as a surrogate parameter. The invention makes use of the fact that a blood vessel is widened to different extents as the blood pressure varies. Since the blood pressure rises briefly and then drops again as a result of a heartbeat, the blood vessel is constantly undergoing periodic deformation. Therefore, the invention is based on the finding that the intensity of the deformation of the blood vessels allows an inference about the prevailing blood pressure, so that an improved blood pressure sensor can be implemented by determining the blood pressure indirectly via the deformation of a blood vessel. Consequently, the surface acoustic wave transponder is preferably designed as a bending beam.

The surface acoustic wave transponder at the same time allows the vasomotor quantity to be determined and read out without requiring a telemetry unit and thus a power supply by battery or induction. For this reason, the implantable sensor may be used independently of an electromedical implant such as a cardiac pacemaker and has small dimensions which help to prevent an impairment of blood flow. Use of the surface acoustic wave transponder as a sensor in a blood vessel also has the additional advantage that growth of cells covering the sensor does not impair the quality of the measurement. The blood pressure measurement can be calibrated by a reference measurement using traditional means. This reference measurement should be repeated at regular intervals because the measurement conditions can alter ingrowth of the sensor.

The surface acoustic wave transponder is pressed against the wall of the blood vessel because of the two retaining stents attached at the ends of the surface acoustic wave transponder, which is why the blood flow through the blood vessel is only slightly hindered because the lumen of the blood vessel remains essentially free. Furthermore, the entire surface acoustic wave transponder grows into the vascular wall over time, so there is practically no longer a risk of thrombosis, which is also why the aforementioned long-term burden of anticoagulant medication is also eliminated. According to an especially preferred embodiment of the invention, retaining stents made of a biocorrodible material, i.e., a material that can be degraded in the body over time, are also provided. The time during which the retaining stents are degraded as expected should be selected so that complete ingrowth of the surface acoustic wave transponder into the vascular wall is ensured before the retaining stents are degraded and/or are no longer able to affix the surface acoustic wave transponder to the vascular wall. In this embodiment the impairment in blood flow due to the blood pressure sensor is minimized after degradation of the retaining stent.

The use of magnesium or pure iron and biocorrodible basic alloys of the elements magnesium, iron, zinc, molybdenum and tungsten is proposed for the retaining stents. A biocorrodible magnesium alloy is preferred for use here. A biocorrodible magnesium alloy is understood to be a metallic structure whose main component is magnesium. The main component is the alloy component whose amount by weight in the alloy is the greatest. The amount of the main component is preferably more than 50 wt %, in particular more than 70 wt %. The biocorrodible magnesium alloy preferably contains yttrium and other rare earth metals because such an alloy is especially suitable because of its physicochemical properties and high biocompatibility, especially also its degradation products. A magnesium alloy having the composition: rare earth metals 5.2-9.9 wt %, including yttrium 3.7-5.5 wt % and remainder <1 wt %, with magnesium accounting for the remainder of the alloy up to 100 wt %, is especially preferred. This magnesium alloy has already confirmed its special suitability experimentally and in preliminary clinical trials, i.e., it has a high biocompatibility, favorable processing properties, good mechanical characteristics and a corrosion behavior that is adequate for the intended purposes.

The composition of the magnesium alloy is to be selected so that it is biocorrodible. The preferred test medium for use in testing the corrosion behavior of alloys is artificial plasma such as that stipulated for biocorrosion tests according to EN ISO 10993-15:2000 (composition NaCl 6.8 g/L, CaCl₂ 0.2 g/L, KCl 0.4 g/L, MgSO₄ 0.1 g/L, NaHCO₃ 2.2 g/L, Na₂HPO₄ 0.126 g/L, NaH₂PO₄ 0.026 g/L). A sample of the material to be tested is therefore stored in a sealed sample container with a defined amount of the test medium at 37° C. At intervals of time from a few hours up to several months, depending on the expected corrosion behavior, the samples are removed and tested for corrosion traces in a known way. The artificial plasma according to EN ISO 10993-15:2000 corresponds to a medium resembling blood and thus offers a possibility of reproducibly simulating a physiological environment in the sense of the invention.

The surface acoustic wave transponder is read out by means of high-frequency query pulses. The query pulses are received by an antenna of the surface acoustic wave transponder and converted into a mechanical surface wave by the force action of the electromagnetic waves received. The surface wave propagates in the surface acoustic wave transponder at a rate of propagation that is lower by several orders of magnitude than that of the query pulse in the medium of air. Therefore, all reflections of the query pulse on surrounding obstacles will have subsided before the surface wave is converted back into an electromagnetic wave due to the piezoelectric effect after traveling through the transponder (optionally after reflection on its other end) and is emitted by the antenna. The emitted signal is weaker by several orders of magnitude than the query pulse but the echoes of the query pulse have already subsided by the time of the response of the surface acoustic wave transponder, so the response of the surface acoustic wave transponder can be received with suitable receivers.

The surface wave propagates in the surface acoustic wave transponder as a function of parameters such as the temperature and mechanical deformation and is influenced by them, which is why inferences regarding these parameters can also be drawn from the response of the surface acoustic wave transponder. Since a virtually constant temperature of approximately 37° C. prevails in a blood vessel based on principle, the surface wave is influenced mainly by the deformation of the surface acoustic wave transponder due to the force of the blood vessel, which expands and contracts again with each beat of the heart, and this is reflected in a variation in the transit times in particular. Therefore, the blood pressure can be determined from the delay in the response of the surface acoustic wave transponder with respect to the query pulse. In addition, reflectors which produce a partial reflection of the surface wave may be applied to the surface acoustic wave transponder. Each partial reflection produces its own response pulse of the surface acoustic wave transponder, with the position of the response pulses in time relative to one another being determined by the spatial arrangement of the reflectors on the surface acoustic wave transponder. Consequently, the change in the interval between two or more response pulses may also be used as a measured value. Furthermore, additional reflectors may also be applied to the surface acoustic wave transponder, where the spatial arrangement of the reflectors forms a characteristic response mark. Such an arrangement is known from the field of RFID transponders. One embodiment of the invention having such additional reflectors has the advantage that automatic identification of the implanted blood pressure sensor and thus during the use of the respective patient is possible, so that measured data of past blood pressure measurements of this patient, for example, may be used automatically for comparison of the current measured data and optionally visualized on a display screen, and the current blood pressure data may be stored in such a way that they are assigned to the patient. The characteristic response mark or a part thereof also makes it possible to use signal technology to separate the response signal more easily from the background noise which always exists when the response mark is known before performing the measurement. If a reflector is positioned on the surface acoustic wave transponder so as to yield the greatest possible interval of time between the point in time of the reflection of the surface wave traveling in the forward direction and the returning surface wave reflected on the reflector at the end of the surface acoustic wave transponder, the determination of the measured quantity may be made on the basis of the relative position of the response pulses generated in passing through the reflector and through the reflected surface wave. To this end, a preferred embodiment of the invention has one or more reflectors, of which at least one first reflector is arranged on a carrier of the surface acoustic wave transponder with a first distance from a pair of interdigital converters and with a second distance from the end of the surface acoustic wave transponder opposite a pair of interdigital converters, such that the second distance is greater than the first distance. The second distance is preferably at least twice as long as the first distance, or better yet, is five or ten times longer than the first distance.

The readout of the surface acoustic wave transponder may take place up to a thousand times per second. Since a heartbeat occurs at the rate of approximately one beat per second, the measurement results collected over a period of 10 to 250 ms, for example, may be averaged to improve the measurement accuracy. Since the propagation rate of the surface wave in the transponder is also much higher than that of the pressure wave caused by the heartbeat in the blood vessel, the variations in blood pressure between two heartbeats over time can be determined, so that the systolic and diastolic blood pressure can easily be determined from the transient measurement.

To ensure better tissue tolerability, the surface acoustic wave transponder is preferably provided with a biocompatible coating. The biocompatible coating is preferably polyurethane or parylene.

In one embodiment variant of the invention, the antenna(s) required for receiving the query pulse and transmitting the response is/are especially advantageously integrated into the implantable sensor, at least one of the retaining stents comprising or functioning as an antenna. The antenna may advantageously be embodied as a frame antenna, which is spanned by the retaining stent in the expansion of the retaining stent. It is likewise possible to subdivide the retaining stent(s) by electrically insulated regions into half-circle or quarter-circle shells or segments, which then form two halves of a half-wave dipole antenna. Two halves of a half-wave dipole antenna may also be applied to the retaining stent, so that the half-wave dipole antenna is spanned by the retaining stent in expansion.

In one embodiment with biocorrodible retraining stents, the antenna is made of a non-biocorrodible material.

Alternatively or additionally, the implantable sensor may have a dipole antenna, such that a longitudinal extent direction of the dipole antenna runs along a connecting line between the retaining stents. The dipole antenna then advantageously fits into the sensor in spatial terms and is aligned at least approximately parallel to the direction of flow of the blood, so that the blood flow is only minimally hindered.

The retaining stents are preferably embodied as self-expanding stents. Alternatively, the retaining stents may also be embodied as balloon-expanded stents.

The implantable sensor may be provided with a marker which allows easy location by means of X-ray or MRT.

### BRIEF DESCRIPTION OF THE DRAWINGS

The invention will now be explained in greater detail on the basis of two figures, in which

FIG. 1 shows a schematic diagram of a surface acoustic wave transponder and

FIG. 2 shows an implantable sensor according to the teaching of the invention.

### DETAILED DESCRIPTION OF THE INVENTION

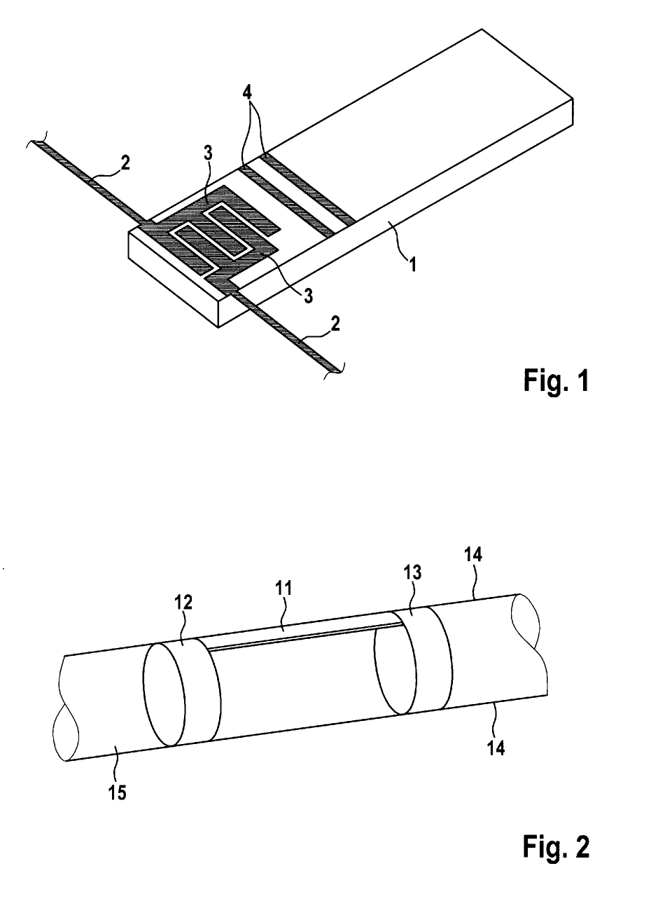

FIG. 1 shows a schematic diagram of a surface acoustic wave transponder. At one end of the carrier 1, interdigital converters 3 connected to an antenna 2 are applied to a carrier 1 consisting of a piezoelectric single crystal; these interdigital converters 3 have a toothed structure and generate a surface wave in the carrier 1 based on the force of the electromagnetic field which occurs on reception of a query pulse via the antenna 2 and acts on the piezoelectric material of the carrier 1. The antenna 2 here is embodied as a divided half-wave dipole, so the interdigital converters 3 pick up the received signal at the center of the antenna 2. The surface wave passes through the carrier 1, where it is exposed to influences due to changes in the path length due to deformation of the carrier 1 and based on elastic crystal constants. Two reflectors 4 are mounted on the carrier 1, generating reflections of the surface wave with a certain interval between them based on the fixed distance between them. In the example illustrated here, two reflectors 4 are arranged close together and close to the interdigital converters 3, so that the surface wave passes the reflectors 4 once before reflection and once after reflection at the end of the carrier 1 opposite the interdigital converters 3. Because of the reflection on the reflectors 4, a characteristic mark is generated by two responses pulses which follow one another in close succession and can also be detected easily even with background noise. The two response pulses follow one another so closely that there is only a minimal influence of the measured quantity on the interval of the response pulses, so this is almost constant. If the rate of propagation of the surface wave is altered by the carrier 1 due to deformation of the carrier 1 by the blood vessel, this yields a corresponding influence on the surface wave which is reflected in the propagation rate of the surface wave and thus in the interval of the pair of response pulses to the response pulse caused by the returning reflected surface wave. Since the surface wave was reflected at the end of the carrier 1 and has traveled back to the interdigital converter 3, the interdigital converter 3 converts the acoustic surface wave into an electromagnetic signal, which is radiated via the antenna 2. Since this emitted signal allows an inference regarding the conditions in the carrier 1 during the propagation of the surface wave, the surface acoustic wave transponder functions as a sensor and allows the detection of fluidic measured quantities, which can serve as surrogate parameters for the blood pressure and blood flow. The reflectors 4 are optional because the point in time of transmission of the query pulse is known in the query device and thus the interval from a single response pulse which is obtained after passing through the carrier 1 twice can be determined. However, the embodiment shown in FIG. 1 with reflectors 4 has the advantage that the transit time of the query and response pulses, which varies with the distance between the query device and the position of the surface acoustic wave transponder, is eliminated from the measurement, thereby increasing the measurement accuracy.

It is also possible to provide two pairs of antennas 2 and interdigital converters 3 which are arranged at the two opposite ends of the carrier 1. In this case, the surface wave is converted back into an electromagnetic signal after passing through the carrier 1 only once and is emitted. This embodiment has the disadvantage that the surface wave is subjected to the influence by the measured quantity in the carrier 1 only once and thus is influenced to a lesser extent. Furthermore, the response of the transponder is obtained in half the time because the path in the transponder is not doubled as it is in the embodiment with just one antenna 2.

FIG. 2 shows an implantable sensor according to the teaching of the invention. A surface acoustic wave transponder 11 is connected at its two narrow ends to just one retaining stent 12, 13 each. The retaining stents 12, 13 are shown in an expanded state in which they are in contact with the vascular wall 14 over the full circumference and thus anchor the implantable sensor in the blood vessel. Since the retaining stents 12, 13 are embodied in a ring shape, the vascular lumen 15 remains free, so that the blood flow is only minimally impaired. Over time, a layer known as neointima is formed on the implantable sensor, this layer being bordered luminally by a monocellular endothelial layer, so that the surface acoustic wave transponder grows completely into the vascular wall over time.

What is claimed is:

1. An implantable sensor for measuring fluidic parameters comprising a surface acoustic wave transponder for detection of vasomotor quantities and one retaining stent attached to each of two opposite ends of the surface acoustic wave transponder.

2. The implantable sensor according to claim 1, wherein the surface acoustic wave transponder is embodied as a bending beam.

3. The implantable sensor according to claim 1, wherein the surface acoustic wave transponder is provided with a biocompatible coating.

4. The implantable sensor according to claim 3, wherein the biocompatible coating is polyurethane.

5. The implantable sensor according to claim 1, wherein at least one of the retaining stents comprises an antenna or is designed to function as an antenna.

6. The implantable sensor according to claim 5, wherein the antenna is a frame antenna and wherein at least one retaining stent spans the frame antenna on expansion of the at least one retaining stent.

7. The implantable sensor according to claim 5, wherein the at least one retaining stent has two electrically insulated sections designed to function as halves of a half-wave dipole antenna.

8. The implantable sensor according to claim 1, wherein the retaining stents are self-expanding stents.

9. The implantable sensor according to claim 1, wherein the retaining stents are balloon-expanded stents.

10. The implantable sensor according to claim 1, wherein the retaining stents are at least partially made of a biocorrodible material.

11. The implantable sensor according to claim 10, wherein the biocorrodible material is a magnesium alloy.

12. The implantable sensor according to claim 1 which has a marker.

13. The implantable sensor according to claim 1, wherein the surface acoustic wave transponder has one or more reflectors.

14. The implantable sensor according to claim 13, wherein at least one first reflector is arranged on a carrier of the surface acoustic wave transponder with a first distance from a pair of interdigital converters and with a second distance from one end of the surface acoustic wave transponder opposite the pair of interdigital converters, the second distance being greater than the first distance.

15. The implantable sensor according to claim 14, wherein the second distance is at least twice as great, five times as great or ten times as great as the first distance.
